(19) United States
(12) Patent Application Publication
(10) Pub. No.: US 2009/0215084 A1
(43) Pub. Date: Aug. 27, 2009

(54) B7-H1 AND B7-H4 IN CANCER

(75) Inventors: Eugene D. Kwon, Rochester, MN (US); John C. Cheville, Pine Island, MN (US); Amy Krambeck, Rochester, MN (US); Susan Kuntz, Rochester, MN (US); Robert H. Thompson, Rochester, MN (US); Lieping Chen, Sparks Glencoe, MD (US); Haidong Dong, Rochester, MN (US); Christine M. Lohse, Rochester, MN (US)

Correspondence Address: FISH & RICHARDSON P.C., PO BOX 1022, MINNEAPOLIS, MN, 55440-1022, US

(73) Assignee: Mayo Foundation for Medical Education and Research

(21) Appl. No.: 12/160,012

(22) PCT Filed: Jan. 5, 2007
(86) PCT No.: PCT/US07/60150

§ 371 Date: Feb. 11, 2009

Related U.S. Application Data
(60) Provisional application No. 60/756,907, filed on Jan. 5, 2006.

(51) Int. Cl. G01N 33/567; C12Q 1/02

(52) U.S. Cl. 435/7.21; 435/29

(57) ABSTRACT

Methods of determining prognosis of a subject with cancer or determining risk of cancer progression by assessing expression of B7-H4, or B7-H1 and B7-H4 in combination.

### CROSS-REFERENCE TO RELATED APPLICATIONS

This application claims the benefit of priority from U.S. Provisional Application Ser. No. 60/756,907, filed Jan. 5, 2006.

### TECHNICAL FIELD

This invention relates to expression of B7-H1 and B7-H4 in biological samples, and more particularly, to using the expression of B7-H4, or B7-H1 and B7-H4 in combination, to determine the prognosis of a subject with cancer or to determine risk of cancer progression in a subject with cancer.

### BACKGROUND

The incidence of renal cell carcinoma (RCC) has increased steadily over the last three decades, with mortality rates continuing to rise. Jemal et al. (2005) CA Cancer J. Clin. 55, 10-30. To date, the only acceptable treatment for clinically localized RCC is surgical extirpation. Improvements in imaging technology have led to a stage migration and with accompanying surgical advancements, improvements in patient survival have been noted. Pantuck et al. (2001) J. Urol. 166, 1611-1623. The five-year survival of RCC patients, however, is still unacceptably low. This low survival rate reflects the 30% of patients who present with metastatic disease, and another 25-30% of patients who subsequently develop disseminated disease after surgical excision of the primary tumor. Motzer et al. (1996) N. Engl. J. Med. 335, 865-875; and Leibovich et al. (2003) Cancer. 97, 1663-1671. Other treatment modalities for advanced disease such as chemotherapy and radiation have not been shown to be effective. Immunotherapy is the only adjunct therapy available, but less than 10% of patients benefit with durable responses. Fyfe et al. (1995) J. Clin. Oncol. 13, 688-696. Limited therapeutic options have done little to improve the median survival of 6-10 months seen in metastatic disease. Figlin et al. (1997) J. Urol. 158, 740-750. Since a large percent of patients with clinically localized disease subsequently develop metastasis, there is a need for prognostic biomarkers.

### SUMMARY

The present application is based in part on the discovery that expression of B7-H4 or co-expression of B7-H1 and B7-H4 in tumors can be used as prognostic biomarkers for clear cell RCC. As described herein, patients who have tumors that are positive for B7-H4, or both B7-H1 and B7-H4, are at an increased risk of cancer progression and death.

In one aspect, the present application features a method of determining the prognosis of a subject with cancer (e.g., RCC). The method includes providing a tissue sample from the subject (e.g., a human); and assessing in the tissue sample the presence or absence of expression of B7-H1 and B7-H4, wherein the presence of expression of B7-H1 and B7-H4 in the tissue sample indicates the subject is more likely to die of the cancer than if only B7-H1 or B7-H4 is expressed in the tissue sample or neither B7-H1 or B7-H4 is expressed in the tissue sample. Expression can be assessed by detecting the presence or absence of polypeptide. For example, detecting can include contacting the tissue sample with an antibody that binds to B7-H1 and an antibody that binds to B7-H4. Each antibody can be fluorescently labeled. Detecting also can include fluorescence flow cytometry (FFC) or immunohistochemistry. The tissue sample can be selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. Renal tissue is particularly useful.

The present application also features a method of determining risk of cancer progression in a subject with cancer. The method includes providing a tissue sample from the subject; and assessing in the tissue sample the presence or absence of expression of B7-H1 and B7-H4, wherein the presence of expression of B7-H1 and B7-H4 in the tissue sample indicates the subject is at more risk of cancer progression than if only B7-H1 or B7-H4 is expressed in the tissue sample or neither B7-H1 or B7-H4 is expressed in the tissue sample. Expression can be assessed by detecting the presence or absence of polypeptide. For example, detecting can include contacting the tissue sample with an antibody that binds to B7-H1 and an antibody that binds to B7-H4. Each antibody can be fluorescently labeled. Detecting also can include FFC or immunohistochemistry. The tissue sample can be selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. Renal tissue is particularly useful.

In another aspect, the present application features an article of manufacture that includes a first antibody that binds to a B7-H1 polypeptide and a second antibody that binds to a B7-H4 polypeptide. The first antibody can be labeled with a first label and the second antibody can be labeled with a second label, wherein the first and second labels are different. The first and second labels can be fluorescent labels.

In yet another aspect, the present application features a method of determining the prognosis of a subject with cancer (e.g., RCC). The method includes providing a tissue sample from the subject (e.g., a human); and assessing in the tissue sample the presence or absence of expression of B7-H4, wherein the presence of expression of B7-H4 in the tissue sample indicates the subject is more likely to die of the cancer than if B7-H4 expression is absent in the tissue sample. Detecting can include contacting the tissue sample with an antibody that binds to B7-H4. The antibody can be fluorescently labeled. The tissue sample can be selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. Renal tissue is particularly useful.

Unless otherwise defined, all technical and scientific terms used herein have the same meaning as commonly understood by one of ordinary skill in the art to which this invention pertains. In case of conflict, the present document, including definitions, will control. Preferred methods and materials are described below, although methods and materials similar or equivalent to those described herein can be used in the practice or testing of the present invention. All publications, patent applications, patents and other references mentioned herein are incorporated by reference in their entirety. The materials, methods, and examples disclosed herein are illustrative only and not intended to be limiting.

Other features and advantages of the invention will be apparent from the following description, from the drawings and from the claims.

### DESCRIPTION OF DRAWINGS

FIG. 1 is a graph depicting the association of tumor B7-H4 expression with cancer-specific survival for 259 patients with clear cell RCC. The cancer-specific survival rates (SE, number still at risk) at 1, 2, and 3 years following nephrectomy were 88.4% (2.6%, 127), 78.5% (3.6%, 83), and 71.2% (4.4%, 43), respectively, for patients with B7-H4-positive tumors compared with 97.1% (1.6%, 99), 90.5% (3.0%, 65), and 90.5% (3.0%, 45), respectively, for patients with B7-H4-negative tumors (p=0.002).

FIG. 2 is a graph depicting the association of combined tumor B7-H1 and B7-H4 expression with cancer-specific survival for 259 patients with clear cell RCC. The cancer-specific survival rates (SE, number still at risk) at 1, 2, and 3 years following nephrectomy were 85.9% (3.6%, 77), 70.9% (5.0%, 52), and 60.9% (5.8%, 27), respectively, for patients with B7-H1-positive and B7-H4-positive tumors compared with 95.6% (1.6%, 149), 91.1% (2.4%, 96), and 91.1% (2.4%, 61), respectively, for patients with negative or singly positive tumors (p<0.001).

### DETAILED DESCRIPTION

In general, the present application provides methods and materials for determining the prognosis of patients with cancer based on the presence or absence of expression of B7-H1 and B7-H4. As used herein, the term “B7-H1” refers to B7-H1 from any mammalian species and the term “hB7-H1” refers to human B7-H1. Further details on B7-H1 polypeptides and nucleic acids are provided in U.S. Pat. No. 6,803,192 and co-pending U.S. application Ser. No. 09/649,108, the disclosures of which are incorporated herein by reference in their entirety. The nucleotide and amino acid sequences of hB7-H1 can be found in GenBank under Accession Nos. AF177937 and AAF25807, respectively. B7-H1 (also known as PD-L1) is a negative regulator of T cell-mediated immunity. See, Dong et al. (1999) Nat. Med. 5, 1365-1369; Dong et al. (2002) Nat. Med. 8, 793-800; and Thompson et al. (2004) Proc. Natl. Acad. Sci. USA 101, 17174-17179. This molecule is constitutively expressed on macrophage-lineage cell surfaces and is expressed in multiple human malignancies. Expression of B7-H1 in normal, non-activated mammalian cells is largely, if not exclusively, limited to macrophage-lineage cells and provides a potential costimulatory signal source for regulation of T cell activation. In contrast, aberrant expression of B7-H1 by tumor cells has been implicated in impairment of T cell function and survival, resulting in defective host antitumoral immunity.

As used herein, the term “B7-H4” refers to B7-H4 from any mammalian species and the term “hB7-H4” refers to human B7-H4. Further details on B7-H4 polypeptides and nucleic acids are provided in U.S. Pat. No. 6,891,030, the disclosure of which is incorporated herein by reference in its entirety. The nucleotide and amino acid sequences of hB7-H4 can be found in GenBank under Accession Nos. AY280972 and AAP37283, respectively. B7-H4 is a negative regulator of T cell-mediated immunity. While the B7-H4 mRNA appears to be constitutively expressed in most tissues, expression of the protein appears to be tightly controlled as B7-H4 protein is not detected in most normal human tissues but has been shown to be sporadically expressed in distal convoluted tubules of the kidney, ductal and acinar cells of the pancreas, endometrial glands, transitional epithelium of the ureter and bladder, bronchial epithelium of the lung, and columnar epithelium of the gallbladder See Tringle et al. (2005) Clin. Cancer Res. 11, 1842-1848. B7-H4 protein has been detected, for example, in ovarian, breast, and lung cancers. See, Choi et al. (2003) J. Immunol. 171, 4650-4655.

### Methods of Determining Prognosis or Risk of Cancer Progression

Expression of B7-H4, or B7-H1 and B7-H4 in combination, can be used to determine prognosis of a patient with cancer and determine the risk of cancer progression. In general, the methods provided herein include assessing the expression of B7-H4, or B7-H1 and B7-H4 in combination, in a tissue sample from a subject, and correlating expression of B7-H4, or B7-H1 and B7-H4, with a poor prognosis or increased risk of cancer progression. Suitable subjects can be mammals, including, for example, humans, non-human primates such as monkeys, baboons, or chimpanzees, horses, cows (or oxen or bulls), pigs, sheep, goats, cats, rabbits, guinea pigs, hamsters, rats, gerbils, and mice. A “tissue sample” is a sample that contains cells or cellular material. Typically, the tissue sample is from a tumor, e.g., a resection or biopsy of a tumor.

As described herein, subjects containing tumors in which ≧5% of the cells express B7-H4 are two-times more likely to die from RCC and are three-times more likely to progress to distant metastases, compared with patients having B7-H4-negative tumors. Furthermore, subjects containing tumors in which <5% of the tumor cells express B7-H1 and B7-H4, or tumors in which ≧5% of the tumor cells express only one of B7-H1 and B7-H4, are less likely to progress to distant metastases or die of the cancer than a subject with the same cancer but in which 5% or more of the tumor cells express B7-H1 and B7-H4.

In particular, with respect to RCC, patients with B7-H1-positive and B7-H4-positive tumors are nearly four times more likely to die from RCC than patients with negative or only singly-positive tumors. As such, prognosis of patients and risk of cancer progression can be determined, at least in part, by assessing the expression of B7-H4, or B7-H1 and B7-H4 in combination. Other factors that can be considered include, for example, the overall health of the patient and previous responses to therapy. Furthermore, assessing expression of B7-H4 and B7-H4 can provide valuable clues as to the course of action to be undertaken in treatment of the cancer, as expression of B7-H1 and B7-H4 indicates a particularly aggressive course of cancer.

Since a number of cancers express B7-H1 and B7-H4, the methods provided herein are applicable to a variety of cancers, including, for example, renal cancer, hematological cancer (e.g., leukemia or lymphoma), neurological cancer, melanoma, breast cancer, lung cancer, head and neck cancer, gastrointestinal cancer, liver cancer, pancreatic cancer, genitourinary cancer, bone cancer, and vascular cancer. As such, suitable tissue samples for assessing B7-H1 and B7-H4 expression can include, for example, lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue. For example, renal, breast, ovarian, and lung tissue samples are particularly useful for determining the prognosis of a patient with RCC, breast, ovarian, or lung cancer, respectively.

In some embodiments, expression of B7-H4, or B7-H1 and B7-H4, can be tested in leukocytes present in any of the above-listed tissues. Leukocytes infiltrating the tissue can be T cells (CD4+ T cells and/or CD8+ T cells) or B lymphocytes. Such leukocytes can also be neutrophils, cosinophils, basophils, monocytes, macrophages, histiocytes, or natural killer cells.

Methods of assessing B7-H1 and B7-H4 expression (RNA and/or polypeptide) can be quantitative, semi-quantitative, or qualitative. Thus, in some embodiments, the level of B7-H1 and B7-H4 expression can be determined as a discrete value. For example, where quantitative RT-PCR is used, the level of expression of B7-H1 mRNA can be measured as a numerical value by correlating the detection signal derived from the quantitative assay to the detection signal of a known concentration of: (a) B7-H1 nucleic acid sequence (e.g., 137-H1 cDNA or B7-H1 transcript); or (b) a mixture of RNA or DNA that contains a nucleic acid sequence encoding B7-H1. Alternatively, the level of B7-H1 or B7-H4 expression can be assessed using any suitable semi-quantitative/qualitative method, including any of a variety of semi-quantitative/qualitative systems known in the art. Thus, the level of expression of B7-H1 or B7-H4 in a cell or tissue sample can be expressed as, for example, (a) one or more of “excellent”, “good”, “satisfactory”, “unsatisfactory”, and/or “poor;” (b) one or more of “very high”, “high”, “average”, “low”, and/or “very low”; or (c) one or more of “++++”, “++++”, “++”, “+”, “+/−”, and/or “−”. Where it is desired, the level of expression of B7-H1 or B7-H4 in tissue from a subject can be expressed relative to the expression of B7-H1 or B7-H4 from (a) a tissue of a subject known not be cancerous (e.g., a contralateral kidney or lung, or an uninvolved lymph node); or (b) a corresponding tissue from one or more other subjects known not to have the cancer of interest, or known not to have any cancer.

Typically, the presence or absence of B7-H1 and B7-H4 expression is determined based on protein expression. As used herein, with respect to B7-H1 and protein expression, the term “presence” indicates that ≧5% of the cells in the tissue sample have detectable levels of B7-H1 and “absence” indicates that <5% of the cells in the tissue sample have detectable levels of B7-H1. With respect to B7-H4 and protein expression, the term “presence” indicates that ≧5% of the cells in the tissue sample have detectable levels of B7-H4 and the term “absence” indicates that <5% of the cells have detectable levels of B7-H4. In some embodiments, however, expression can be based on mRNA levels. In other embodiments, the level of expression of B7-H4, or B7-H1 and B7-H4, in tissue from a subject can be expressed relative to the expression of B7-H4, or B7-H1 and B7-H4 from (a) a tissue of a subject known not be cancerous (e.g., a contralateral kidney or lung, or an uninvolved lymph node); or (b) a corresponding tissue from one or more other subjects known not to have the cancer of interest, or known not to have any cancer.

Any suitable method can be used to detect expression of a protein in a tissue sample, including those known in the art. For example, antibodies that bind to an epitope specific for B7-H1 can be used to assess the presence or absence of B7-H1 expression, and antibodies that bind to an epitope specific for B7-H4 can be used to assess the presence or absence of B7-H4 expression. As used herein, the terms “antibody” or “antibodies” include intact molecules (e.g., polyclonal antibodies, monoclonal antibodies, humanized antibodies, or chimeric antibodies), as well as fragments thereof (e.g., single chain Fv antibody fragments, Fab fragments, and F(ab)₂ fragments), that are capable of binding to an epitopic determinant of B7-H1 or B7-H4 (e.g., hB7-H1 or hB7-H4). The term “epitope” refers to an antigenic determinant on an antigen to which the paratope of an antibody binds. Epitopic determinants usually consist of chemically active surface groupings of molecules such as amino acids or sugar side chains, and typically have specific three-dimensional structural characteristics, as well as specific charge characteristics. Epitopes generally have at least five contiguous amino acids (a continuous epitope), or alternatively can be a set of noncontiguous amino acids that define a particular structure (e.g., a conformational epitope). Polyclonal antibodies are heterogeneous populations of antibody molecules that are contained in the sera of the immunized animals. Monoclonal antibodies are homogeneous populations of antibodies to a particular epitope of an antigen.

Antibody fragments that can bind to B7-H1 or B7-H4 can be generated using any suitable technique, including those known in the art. For example, F(ab′)₂ fragments can be produced by pepsin digestion of the antibody molecule; Fab fragments can be generated by reducing the disulfide bridges of F(ab′)₂ fragments. Alternatively, Fab expression libraries can be constructed. See, for example, Huse et al. (1989) Science, 246, 1275. Once produced, antibodies or fragments thereof can be tested for recognition of B7-H1 or B7-H4 using standard immunoassay methods, including ELISA techniques, radioimmunoassays, and Western blotting. See, Short Protocols in Molecular Biology, Chapter 11, Green Publishing Associates and John Wiley & Sons, Edited by Ausubel, F. M et al., 1992.

Antibodies having specific binding affinity for B7-H1 or B7-H4 can be produced using, for example, standard methods. See, e.g., Dong et al. (2002) Nature Med. 8, 793-800. In general, a B7-H1 or B7-H4 polypeptide can be recombinantly produced, or can be purified from a biological sample, and used to immunize animals. As used herein, the term “polypeptide” refers to a polypeptide of at least five amino acids in length. To produce a recombinant B7-H1 or B7-H4 polypeptide, a nucleic acid sequence encoding the appropriate polypeptide can be ligated into an expression vector and used to transform a bacterial or eukaryotic host cell. Nucleic acid constructs typically include a regulatory sequence operably linked to a B7-H1 or B7-H4 nucleic acid sequence. Regulatory sequences do not typically encode a gene product, but instead affect the expression of the nucleic acid sequence. In bacterial systems, a strain of Escherichia coli such as BL-21 can be used. Suitable E. coli vectors include the pGEX series of vectors that produce fusion proteins with glutathione S-transferase (GST). Transformed E. coli are typically grown exponentially, then stimulated with isopropylthiogalactopyranoside (IPTG) prior to harvesting. In general, such fusion proteins are soluble and can be purified easily from lysed cells by adsorption to glutathione-agarose beads followed by elution in the presence of free glutathione. The pGEX vectors are designed to include thrombin or factor Xa protease cleavage sites so that the cloned target gene product can be released from the GST moiety.

Mammalian cell lines that stably express a B7-H1 or B7-H4 polypeptide can be produced by using expression vectors with the appropriate control elements and a selectable marker. For example, the eukaryotic expression vector pCDNA.3.1+(Invitrogen, San Diego, Calif.) can be used to express a B7-H1 or B7-H4 polypeptide in, for example, COS cells, Chinese hamster ovary (CHO), or HEK293 cells. Following introduction of the expression vector by electroporation, DEAE dextran, or other suitable method, stable cell lines can be selected. Alternatively, B7-H1 or B7-H4 can be transcribed and translated in vitro using wheat germ extract or rabbit reticulocyte lysate.

In eukaryotic host cells, a number of viral-based expression systems can be utilized to express a B7-H1 or B7-H4 polypeptide. A nucleic acid encoding a B7-H1 or B7-H4 polypeptide can be introduced into an SV40, retroviral or vaccinia based viral vector and used to infect host cells. Alternatively, a nucleic acid encoding a B7-H1 or B7-H4 polypeptide can be cloned into, for example, a baculoviral vector and then used to transfect insect cells.

Various host animals can be immunized by injection of the B7-H1 or B7-H4 polypeptide. Host animals can include rabbits, chickens, mice, guinea pigs and rats. Various adjuvants that can be used to increase the immunological response depend on the host species, and include Freund's adjuvant (complete and incomplete), mineral gels such as aluminum hydroxide, surface-active substances such as lysolecithin, pluronic polyols, polyanions, peptides, oil emulsions, keyhole limpet hemocyanin and dinitrophenol. Monoclonal antibodies can be prepared using a B7-H1 or B7-H4 polypeptide and standard hybridoma technology. In particular, monoclonal antibodies can be obtained by any technique that provides for the production of antibody molecules by continuous cell lines in culture such as described by Kohler et al. (1975) Nature, 256, 495, the human B-cell hybridoma technique (Kosbor et al. (1983) Immunology Today, 4, 72; Cote et al. (1983) Proc. Natl. Acad. Sci. USA, 80, 2026), and the EBV-hybridoma technique (Cole et al., “Monoclonal Antibodies and Cancer Therapy”, Alan R. Liss, Inc., pp. 77-96 (1983)). Such antibodies can be of any immunoglobulin class, including IgG, IgM, IgE, IgA, IgD, and any subclass thereof. The hybridoma producing the monoclonal antibodies provided herein can be cultivated in vitro and in vivo.

In immunological assays, an antibody having specific binding affinity for B7-H1 or B7-H4, or a secondary antibody that binds to an antibody having specific binding affinity for B7-H1 or B7-H4, can be labeled, either directly or indirectly. Suitable labels include, without limitation, radionuclides (e.g., ¹²⁵I, ¹³¹I, ³⁵S, ³H, ³²P, ³³P, or ¹⁴C), fluorescent moieties (e.g., fluorescein, fluorescein-5-isothiocyanate (FITC), PerCP, rhodamine, or phycoerythrin), luminescent moieties (e.g., Qdot™ nanoparticles supplied by the Quantum Dot Corporation, Palo Alto, Calif.), compounds that absorb light of a defined wavelength, or enzymes (e.g., alkaline phosphatase or horseradish peroxidase). Antibodies also can be indirectly labeled by conjugation with biotin and then detected with avidin or streptavidin labeled with a molecule as described above. In embodiments in which antibodies to B7-H1 and B7-H4 are used in combination, the antibodies can be labeled such that each can be distinctly visualized (e.g., by labeling with two different fluorescent moieties). Methods of detecting or quantifying a label depend on the nature of the label, and include those known in the art. Examples of detectors include, without limitation, x-ray film, radioactivity counters, scintillation counters, spectrophotometers, calorimeters, fluorometers, luminometers, and densitometers. Combinations of these approaches (including “multi-layer” assays) familiar to those in the art can be used to enhance the sensitivity of assays.

Immunological assays for detecting B7-H1 or B7-H4 can be performed in a variety of known formats, including sandwich assays (e.g., ELISA assays, sandwich Western blotting assays, or sandwich immunomagnetic detection assays), competition assays (competitive RIA), or bridge immunoassays. See, for example, U.S. Pat. Nos. 5,296,347; 4,233,402; 4,098,876; and 4,034,074. Some protein-detecting assays (e.g., ELISA or Western blot) can be applied to lysates of cells, and others (e.g., immunohistological methods or fluorescence flow cytometry) can be applied to histological sections or unlysed cell suspensions.

In other embodiments, the presence or absence of B7-H1 expression can be determined based on mRNA levels. As used herein with respect to mRNA expression, the term “presence” indicates that the tumor sample contains a significantly increased level of mRNA relative to (a) a tissue of a subject known not be cancerous (e.g., a contralateral kidney or lung, or an uninvolved lymph node); or (b) a corresponding tissue from one or more other subjects known not to have the cancer of interest, or known not to have any cancer. As used herein with respect to mRNA expression, the term “absence” indicates that the tumor sample does not contain a significantly increased level of mRNA relative to (a) a tissue of a subject known not be cancerous; or (b) a corresponding tissue from one or more other subjects known not to have the cancer of interest, or known not to have any cancer.

Suitable methods for detecting an mRNA in a tissue sample include, for example, methods known in the art. For example, cells can be lysed and an mRNA in the lysates or in RNA purified or semi-purified from the lysates can be detected by any of a variety of methods including, without limitation, hybridization assays using delectably labeled gene-specific DNA or RNA probes (e.g., Northern Blot assays) and quantitative or semi-quantitative RT-PCR methodologies using appropriate gene-specific oligonucleotide primers. Alternatively, quantitative or semi-quantitative in situ hybridization assays can be carried out using, for example, tissue sections or unlysed cell suspensions, and delectably (e.g., fluorescently or enzyme) labeled DNA or RNA probes. Additional methods for quantifying mRNA include RNA protection assay (RPA) and SAGE.

### Articles of Manufacture

Antibodies that can bind to a B7-H1 polypeptide (e.g., hB7-H1) and antibodies that can bind to a B7-H4 polypeptide (e.g., hB7-H4) can be combined with packaging material and sold as a kit for detecting B7-H1 and B7-H4 from biological samples, determining prognosis of a subject with cancer, or determining risk of cancer progression in a subject. Components and methods for producing articles of manufactures are well known. In addition, the articles of manufacture may further include reagents such as secondary antibodies, sterile water, pharmaceutical carriers, buffers, indicator molecules, solid phases (e.g., beads), and/or other useful reagents (e.g., positive and negative controls) for detecting B7-H1 and B7-H4 from biological samples, determining prognosis of a subject with cancer, or determining risk of cancer progression in a subject. The antibodies can be in a container, such as a plastic, polyethylene, polypropylene, ethylene, or propylene vessel that is either a capped tube or a bottle. In some embodiments, the antibodies can be included on a solid phase such as a handheld device for bedside testing. Instructions describing how the various reagents are effective for determining prognosis of a subject with cancer or determining risk of cancer progression also may be included in such kits.

The invention will be further described in the following examples, which do not limit the scope of the invention described in the claims.

### EXAMPLES

### Example 1

### Materials and Methods

Patient selection—Upon approval from the Mayo Clinic Institutional Review Board, 531 patients were identified from Mayo Clinic Nephrectomy Registry that were previously treated with radical nephrectomy or nephron-sparing surgery for unilateral, sporadic, non-cystic clear cell RCC between 2000 and 2003. Since pathologic features and patient outcome differ by RCC subtype, all analyses were restricted to patients treated with clear cell RCC only, the most common of the RCC subtypes [Cheville et al. (2003) Am. J. Surg. Pathol. 27, 612-624]. In addition, patients were selected based on the availability of fresh-frozen tissue since the human B7-H4 specific monoclonal antibody, hH4.1, can reproducibly stain only fresh-frozen, not paraffin-fixed, tissue during immunohistochemical analysis.

Clinical and Pathologic features—The clinical features studied included age, sex, and symptoms. Patients with a palpable flank or abdominal mass, discomfort, gross hematuria, acute onset varicocele, or constitutional symptoms including rash, sweats, weight loss, fatigue, early satiety, and anorexia were considered symptomatic at presentation. The pathologic features studied included histologic subtype, tumor size, the 2002 primary tumor classification, regional lymph node involvement, distant metastases at nephrectomy, the 2002 TNM stage groupings, nuclear grade, coagulative tumor necrosis, and sarcomatoid differentiation. These features were obtained by a review of the microscopic slides from all nephrectomy specimens by a urologic pathologist, without knowledge of patient outcome.

B7-H1 Immunohistochemical staining of tumor specimens—Cryosections from RCC tumors (5 μm, −20° C.) were mounted on Superfrost Plus slides, air dried, and fixed in ice-cold acetone. Sections were stained using the Dako Autostainer and Dako Cytomation Labeled Polymer (EnVision+) HRP detection kit (Dako; Carpinteria, Calif.). Slides were blocked with H₂O₂ for 10 minutes followed by incubation with the antibody applied for 30 minutes at room temperature. Labeled polymer then was applied at room temperature for 15 minutes followed by incubation with chromogen-substrate for 10 minutes. Finally, sections were counter-stained for three minutes with modified Schmidt's Hematoxylin. The antibody used was the mouse anti-human B7-H1 monoclonal antibody 5H1. Human tonsil tissue was used as a positive control. Irrelevant isotype-matched antibodies were used to control for non-specific staining.

B7-H4 Immunohistochemical Staining—Cryosections from RCC tumors were prepared as described above. Sections were stained using the Dako Autostainer and Dako Cytomation CSA II kit (Dako; Carpinteria, Calif.). Slides were blocked with H₂O₂ for five minutes followed by incubation with the antibody applied for 30 minutes at room temperature. Anti-mouse immunoglobulin-HRP was then applied at room temperature for 15 minutes followed by incubation with amplification reagent for 15 minutes. Slides were then incubated for 15 minutes with anti-fluorescein-HRP and visualized with DAB substrate for 8 minutes. Finally, sections were counter-stained for one minute with Hematoxylin. The antibody used for this protocol was the mouse anti-human B7-H4 monoclonal antibody hH4.1. Human ovarian cancer tissue was used as a positive control. Irrelevant isotype-matched antibodies were used to control for non-specific staining.

Quantification of B7-H1 and B7-H4 Expression—The percentages of tumor cells that stained positive for B7-H1 and B7-H4 were quantified in 5% increments by a urologic pathologist. The tumor was considered positive if there was histologic evidence of cell-surface membrane staining. Cases with <5% tumor staining were considered negative.

Statistical methods—Comparisons among the clinical and pathologic features were evaluated using chi-square and Fisher's exact tests. Overall, cancer-specific, and progression-free survival was estimated using the Kaplan-Meier method. The duration of follow-up was calculated from the date of surgery to the date of cancer progression (i.e., distant metastases), death, or last known follow-up. Cause of death was determined from death certificate or physician correspondence. The associations of B7-H1 and B7-H4 tumor expression with death from any cause, death from RCC, and cancer progression were evaluated using Cox proportional hazards regression models univariately and after adjusting for the Mayo Clinic SSIGN (Stage, SIze, Grade, and Necrosis) Score, a prognostic composite score specifically developed for patients with clear cell RCC. These associations were summarized using risk ratios (RR) and 95% confidence intervals (95% C1). Statistical analyses were performed using the SAS software package (SAS Institute; Cary, N.C.). All tests were two-sided and p-values <0.05 were considered statistically significant.

### Example 2

### Survival of RCC Patients with Fresh-Frozen Tissue Samples Available

Of the 531 patients eligible for study, 259 (49%) had fresh-frozen tissue available for laboratory investigation. None of the clinical or pathologic features studied was significantly different between patients with and without fresh-frozen tissue available for study. Furthermore, there was not a statistically significant difference in overall survival (p=0.739) or cancer-specific survival (p=0.780) between the two groups.

At last follow-up, 63 of the 259 patients studied had died, including 47 patients who died from RCC at a median of 1.2 years following surgery (range 0-4.4). Among the 196 patients who were still alive at last follow-up, the median duration of follow-up was 2.6 years (range 0-5.6). Estimated overall survival rates (standard error [SE], number still at risk) at 1, 2, and 3 years following surgery were 90.3% (1.9%, 226), 79.7% (2.7%, 148), and 73.9% (3.1%, 88), respectively. Cancer-specific survival rates (SE, number still at risk) at the same time points were 92.1% (1.7%, 226), 83.5% (2.5%, 148), and 79.3% (2.9%, 88), respectively. Among the subset of 215 patients with clinically localized RCC at surgery (i.e., pNX/pN0, pM0), 36 progressed to distant metastases at a median of 1.1 years following surgery (range 0-4.9). Progression-free survival rates (SE, number still at risk) at 1, 2, and 3 years following surgery were 91.9% (1.9%, 187), 84.8% (2.6%, 125), and 81.5% (3.0%, 74), respectively.

### Example 3

### Tumor B7-H4 Expression

One hundred fifty-three (59.1%) patients had positive tumor B7-H4 staining, with a median level of staining of 20% (range 5%-90%). A comparison of clinical and pathologic features by tumor B7-H4 expression is shown in Table 1. Positive tumor B7-H4 expression was associated with adverse clinical and pathologic features including the presence of constitutional symptoms, larger tumor size, higher tumor stage and grade, and tumor necrosis. For example, only one (0.9%) patient with a B7-H4-negative tumor had regional lymph node involvement compared with 14 (9.2%) patients with B7-H4-positive tumors (p=0.005).

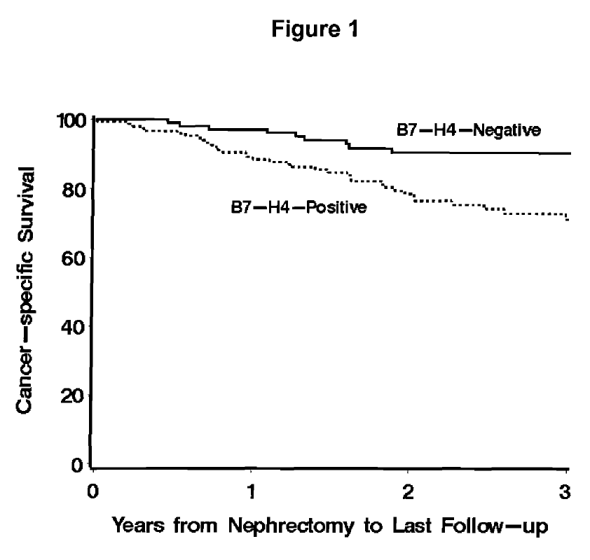

Univariately, patients with B7-H4-positive tumors were over twice as likely to die from any cause compared with patients with B7-H4-negative tumors (risk ratio 2.51; 95% C11.42-4.45; p=0.002). The overall survival rate (SE, number still at risk) at 3 years following surgery for patients with B7-H4-positive tumors was 66.1% (4.5%, 43) compared with 84.5% (3.9%, 45) for patients with B7-H4-negative tumors. Patients with B7-H4-positive tumors also were significantly more likely to die from RCC (risk ratio 3.05; 95% CI 1.51-6.14; p=0.002; FIG. 1). The 3-year cancer-specific survival rates (SE, number still at risk) for patients with B7-H4-positive and B7-H4-negative tumors were 71.2% (4.4%, 43) and 90.5% (3.0%, 45), respectively. After adjusting for the SSIGN Score, patients with B7-H4-positive tumors were still nearly twice as likely to die from RCC, but this difference did not attain statistical significance (risk ratio 1.78; 95% CI 0.88-3.63; p=0.112). Among the subset of 215 patients with clinically localized RCC at surgery, patients with B7-H4-positive tumors were three times more likely to progress compared with patients with B7-H4-negative tumors (risk ratio 2.99; 95% CI 1.36-6.57; p=0.006). The 3-year progression-free survival rate (SE, number still at risk) for patients with B7-H4-positive tumors was 74.1%

TABLE 1
Comparison of Pathologic Features by Tumor B7-H4 Expression

| | Tumor B7-H4 Expression | | |
|---|---|---|---|
| | Negative | Positive | |
| | N = 106 | N = 153 | |
| Feature | N (%) | | P-value |
| Age at Surgery (years) | | | |
| <65 | 55 (51.9) | 81 (52.9) | 0.867 |
| ≧65 | 51 (48.1) | 72 (47.1) | |
| Sex | | | |
| Female | 40 (37.7) | 45 (29.4) | 0.161 |
| Male | 66 (62.3) | 108 (70.6) | |
| Symptoms at Presentation | 49 (46.2) | 86 (56.2) | 0.114 |
| Constitutional Symptoms | 9 (8.5) | 31 (20.3) | 0.010 |
| at Presentation | | | |
| Primary Tumor Size (cm) | | | |
| <5 | 54 (50.9) | 48 (31.4) | <0.001 |
| 5 to <7 | 25 (23.6) | 28 (18.3) | |
| 7 to <10 | 12 (11.3) | 35 (22.9) | |
| ≧10 | 15 (14.2) | 42 (27.5) | |
| 2002 Primary Tumor | | | |
| Classification | | | |
| pT1a | 41 (38.7) | 40 (26.1) | 0.012 |
| pT1b | 32 (30.2) | 29 (19.0) | |
| pT2 | 11 (10.4) | 28 (18.3) | |
| pT3a | 10 (9.4) | 18 (11.8) | |
| pT3b | 11 (10.4) | 32 (20.9) | |
| pT3c | 1 (0.9) | 4 (2.6) | |
| pT4 | 0 (0.0) | 2 (1.3) | |
| Regional Lymph Node | | | |
| Involvement | | | |
| pNX/pN0 | 105 (99.1) | 139 (90.9) | 0.005 |
| pN1/pN2 | 1 (0.9) | 14 (9.1) | |
| Distant Metastases at | | | |
| Nephrectomy | | | |
| pM0 | 91 (85.9) | 128 (83.7) | 0.632 |
| pM1 | 15 (14.1) | 25 (16.3) | |
| 2002 TNM Stage Groupings | | | |
| I | 69 (65.1) | 68 (44.4) | 0.006 |
| II | 10 (9.4) | 20 (13.1) | |
| III | 12 (11.3) | 39 (25.5) | |
| IV | 15 (14.2) | 26 (17.0) | |
| Nuclear Grade | | | |
| 1 | 7 (6.6) | 6 (3.9) | <0.001 |
| 2 | 53 (50.0) | 33 (21.6) | |
| 3 | 42 (39.6) | 89 (58.2) | |
| 4 | 4 (3.8) | 25 (16.3) | |
| Coagulative Tumor Necrosis | 16 (15.1) | 57 (37.3) | <0.001 |
| Sarcomatoid Differentiation | 1 (0.9) | 7 (4.6) | 0.094 |

(4.5%, 34) compared with 91.2% (3.2%, 40) for patients with B7-H4-negative tumors.

### Example 4

### Combination of Tumor B7-H1 and B7-H4 Expression

There were 59 (22.8%) tumors that were both B7-H1-negative and B7-H4-negative, 59 (22.8%) that were B7-H1-negative and B7-H4-positive, 47 (18.2%) that were B7-H1-positive and B7-H4-negative, and 94 (36.3%) that were both B7-H1-positive and B7-H4-positive. Tumors that were B7-H1-positive were more likely to be B7-H4-positive compared with tumors that were B7-H1-negative (66.7% versus 50.0%; p=0.007).

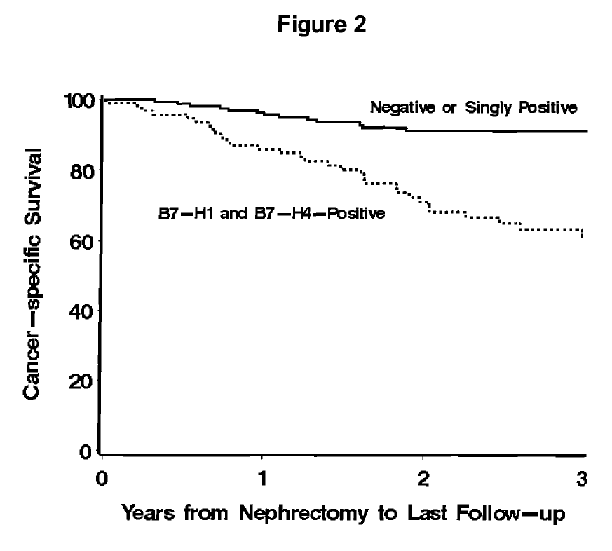

When combined in a model together, positive tumor B7-H1 expression (risk ratio 2.63; 95% CI 1.42-4.86; p=0.002) and positive tumor B7-H4 expression (risk ratio 2.21; 95% CI 1.24-3.93; p=0.007) were independently significantly associated with death from any cause. This was also true for the associations of positive B7-H1 expression (risk ratio 3.95; 95% CI 1.76-8.85; p<0.001) and positive B7-H4 expression (risk ratio 2.57; 95% CI 1.27-5.20; p=0.009) with death from RCC. The 3-year cancer-specific survival rates for patients with B7-H1-negative and B7-H4-negative tumors, B7-H1-negative and B7-H4-positive tumors, B7-H1-positive and B7-H4-negative, and B7-H1-positive and B7-H4-positive tumors were 94.0%, 92.3%, 86.6%, and 60.9%, respectively. Cancer-specific survival rates did not differ significantly among patients in the first three groups (p=0.308). However, cancer-specific survival was significantly lower for patients with B7-H1-positive and B7-H4-positive tumors compared with patients in the other three groups (p<0.001). Patients with B7-H1-positive and B7-H4-positive tumors were over four times more likely to die from RCC compared with patients with negative or only singly positive tumors (risk ratio 4.49; 95% CI 2.40-8.39; p<0.001; FIG. 2); a difference which persisted even after adjusting for the SSIGN Score (risk ratio 3.69; 95% CI 1.95-6.98; p<0.001). In fact, 33 of the 47 patients who died from RCC had tumors that were positive for both B7-H1 and B7-H4. Among the subset of 215 patients with clinically localized RCC at surgery, patients with B7-H1-positive and B7-H4-positive tumors were significantly more likely to

TABLE 2
Comparison of Pathologic Features by Combined
Tumor B7-H1 and B7-H4 Expression

| | B7-H1-Positive and | | |
|---|---|---|---|
| | B7-H4-Positive | | |
| | No | Yes | |
| | N = 165 | N = 94 | |
| Feature | N (%) | | P-value |
| Age at Surgery (years) | | | |
| <65 | 91 (55.2) | 45 (47.9) | 0.259 |
| ≧65 | 74 (44.8) | 49 (52.1) | |
| Sex | | | |
| Female | 54 (32.7) | 31 (33.0) | 0.967 |
| Male | 111 (67.3) | 63 (67.0) | |
| Symptoms at Presentation | 78 (47.3) | 57 (60.6) | 0.038 |
| Constitutional Symptoms | 17 (10.3) | 23 (24.5) | 0.002 |
| at Presentation | | | |
| Primary Tumor Size (cm) | | | |
| <5 | 76 (46.1) | 26 (27.7) | <0.001 |
| 5 to <7 | 41 (24.9) | 12 (12.8) | |
| 7 to <10 | 21 (12.7) | 26 (27.7) | |
| ≧10 | 27 (16.4) | 30 (31.9) | |
| 2002 Primary Tumor | | | |
| Classification | | | |
| pT1a | 62 (37.6) | 19 (20.2) | <0.001 |
| pT1b | 46 (27.9) | 15 (16.0) | |
| pT2 | 16 (9.7) | 23 (24.5) | |
| pT3a | 15 (9.1) | 13 (13.8) | |
| pT3b | 24 (14.6) | 19 (20.2) | |
| pT3c | 2 (1.2) | 3 (3.2) | |
| pT4 | 0 (0.0) | 2 (2.1) | |
| Regional Lymph Node | | | |
| Involvement | | | |
| pNX/pN0 | 161 (97.6) | 83 (88.3) | 0.002 |
| pN1/pN2 | 4 (2.4) | 11 (11.7) | |
| Distant Metastases at | | | |
| Nephrectomy | | | |
| pM0 | 143 (86.7) | 76 (80.9) | 0.213 |
| pM1 | 22 (13.3) | 18 (19.1) | |
| 2002 TNM Stage Groupings | | | |
| I | 103 (62.4) | 34 (36.2) | <0.001 |
| II | 14 (8.5) | 16 (17.0) | |
| III | 26 (15.8) | 25 (26.6) | |
| IV | 22 (13.3) | 19 (20.2) | |
| Nuclear Grade | | | |
| 1 | 12 (7.3) | 1 (1.1) | <0.001 |
| 2 | 71 (43.0) | 15 (16.0) | |
| 3 | 72 (43.6) | 59 (62.8) | |
| 4 | 10 (6.1) | 19 (20.2) | |
| Coagulative Tumor Necrosis | 32 (19.4) | 41 (43.6) | <0.001 |
| Sarcomatoid Differentiation | 2 (1.2) | 6 (6.4) | 0.029 |

progress to distant metastases compared with patients with negative or singly positive tumors (risk ratio 2.58%; 95% CI 1.34-4.99; p = 0.005).

A comparison of clinical and pathologic features by the combination of tumor B7-H1 and B7-H4 expression in shown in Table 2. Patients with the B7-H 1-positive, B7-H4-positive phenotype were significantly more likely to exhibit adverse clinical and pathologic features including symptoms at presentation, larger tumor size, higher tumor stage and grade, tumor necrosis, and sarcomatoid differentiation. For example, 10 (6.1%) patients with negative or singly positive tumors had grade 4 RCC compared with 19 (20.2%) patients with B7-H 1-positive and B7-H4-positive tumors (p<0.001).

### Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. A method of determining the prognosis of a subject with cancer, said method comprising:
(a) providing a tissue sample from said subject; and
(b) assessing in said tissue sample the presence or absence of expression of B7-H1 and B7-H4, wherein the presence of expression of B7-H1 and B7-H4 in said tissue sample indicates said subject is more likely to die of the cancer than if only B7-H1 or B7-H4 is expressed in said tissue sample.

2. The method of claim 1, wherein expression is assessed by detecting the presence or absence of polypeptide.

3. The method of claim 2, wherein detecting comprises contacting said tissue sample with an antibody that binds to B7-H1 and an antibody that binds to B7-H4.

4. The method of claim 3, wherein each said antibody is fluorescently labeled.

5. The method of claim 2, wherein detecting comprises fluorescence flow cytometry (FFC).

6. The method of claim 2, wherein detecting comprises immunohistochemistry.

7. The method of claim 1, wherein said tissue sample is selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue.

8. The method of claim 7, wherein said tissue sample is renal tissue.

9. The method of claim 1, wherein the cancer is renal cell carcinoma.

10. The method of claim 1, wherein said subject is a human.

11. A method of determining risk of cancer progression in a subject with cancer, said method comprising:
(a) providing a tissue sample from said subject; and
(b) assessing in said tissue sample the presence or absence of expression of B7-H1 and B7-H4, wherein the presence of expression of B7-H1 and B7-H4 in said tissue sample indicates said subject is at more risk of cancer progression than if only B7-H1 or B7-H4 is expressed in said tissue sample.

12. The method of claim 11, wherein expression is assessed by detecting the presence or absence of polypeptide.

13. The method of claim 12, wherein detecting comprises contacting said tissue sample with an antibody that binds to the B7-H1 polypeptide and an antibody that binds to B7-H4.

14. The method of claim 12, wherein detecting comprises FFC.

15. The method of claim 12, wherein detecting comprises immunohistochemistry.

16. The method of claim 11, wherein said tissue sample is selected from the group consisting of lung, epithelial, connective, vascular, muscle, neural, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue.

17. The method of claim 16, wherein said tissue sample is renal tissue.

18. The method of claim 11, wherein the subject is a human.

19. An article of manufacture, said article of manufacture comprising a first antibody that binds to a B7-H1 polypeptide and a second antibody that binds to a B7-H4 polypeptide.

20. The article of manufacture of claim 19, wherein said first antibody is labeled with a first label and said second antibody is labeled with a second label, wherein said first and second labels are different.

21. The article of manufacture of claim 20, wherein said first and second labels are fluorescent labels.

22. A method of determining risk of cancer progression in a subject with cancer, said method comprising:
(a) providing a tissue sample from said subject; and
(b) assessing in said tissue sample the presence or absence of expression of B7-H4, wherein the presence of expression of B7-H4 in said tissue sample indicates said subject is at more risk of cancer progression than if B7-H4 expression is absent in said tissue sample.

23. The method of claim 22, wherein detecting comprises contacting said tissue sample with an antibody that binds to B7-H4.

24. The method of claim 23, wherein said antibody is fluorescently labeled.

25. The method of claim 22, wherein said tissue sample is selected from the group consisting of lung, epithelial, connective, vascular, muscle, nervous, skeletal, lymphatic, prostate, cervical, breast, spleen, gastric, intestinal, oral, esophageal, dermal, liver, bladder, thyroid, thymic, adrenal, brain, gallbladder, pancreatic, uterine, ovarian, and testicular tissue.

26. The method of claim 25, wherein said tissue sample is renal tissue.

27. The method of claim 22, wherein the cancer is renal cell carcinoma.

28. The method of claim 22, wherein said subject is a human.
